(19) United States
(12) Patent Application Publication
(10) Pub. No.: US 2024/0300933 A1
(43) Pub. Date: Sep. 12, 2024

(54) SALT AND CRYSTAL FORMS OF AN HMOX1 INDUCER

(71) Applicant: MITOBRIDGE INC., Cambridge, MA (US)

(72) Inventors: Shanming Kuang, Cranbury, NJ (US); Juan Hu, Shanghai (CN); Margaret Biddle, Cambridge, MA (US)

(21) Appl. No.: 18/277,746

(22) PCT Filed: Feb. 18, 2022
(86) PCT No.: PCT/US2022/017009

§ 371 Date: Aug. 17, 2023

(30) Foreign Application Priority Data
Feb. 19, 2021 (WO) PCT/CN2021/076827

(51) Int. Cl. C07D 413/12; A61K 31/423

(52) U.S. Cl. CPC C07D 413/12; A61K 31/423

(57) ABSTRACT

Disclosed is the 1:1 phosphate salt of Compound (I) as well as a novel crystal form thereof. Also disclosed is a novel crystal form of Compound (I). The present invention also provides methods of increasing the activity of HMOX-1 in a subject, of activating transcription factor Nrf2 in a subject or of reducing the amount of ROS in a subject by administering an effective amount of the 1:1 phosphate salt of Compound (I), the crystal form thereof or the crystal form of Compound (I) to the subject.

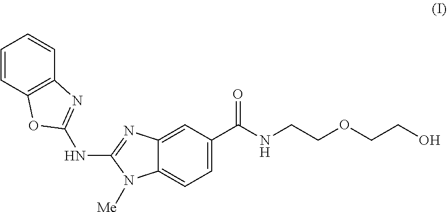 [SMILES: Cn1c(Nc2nc3ccccc3o2)nc2cc(C(=O)NCCOCCO)ccc21]

### REFERENCE TO RELATED APPLICATION

This application claims the benefit of International Application No. PCT/CN2021/076827, filed Feb. 19, 2021, the entire contents of which are incorporated herein by reference.

### FIELD OF THE INVENTION

This application is directed to salt and salt crystal forms of an HMOX1 (heme oxygenase 1) inducer, and methods for their use, such as to control the activity or the amount, or both the activity and the amount, of heme-oxygenase in a mammalian subject.

### BACKGROUND OF INVENTION

Oxidative stress represents an imbalance between cellular reactive oxygen species (ROS) production and cellular responses to ROS such as degrading ROS species and producing endogenous anti-oxidant molecules.

ROS serve critical cellular signaling needs, but can have deleterious effects if overproduced or left unchecked. Increased ROS levels in a cell can result in damage to components such as lipids, proteins, polysaccharides, and DNA. Prolonged oxidative stress is also linked to chronic diseases that affect nearly every major organ system. For example, prolonged oxidative stress is implicated in the onset or progression of disease states such as neurodegenerative diseases, lung diseases, cardiovascular diseases, renal diseases, diabetes, inflammatory pain, and cancer. Accordingly, strategies to mitigate oxidative stress are desirable for a number of therapeutic settings.

Under normal physiological conditions, production of ROS is counterbalanced by a well-defined and conserved set of cellular pathways that respond to, limit, and repair the damage due to ROS. This adaptive set of genes are called the phase II system. They encode enzymes that degrade ROS directly as well as increase levels of cells' endogenous antioxidant molecules, including glutathione and bilirubin.

Of the phase II enzyme system, HMOX1, a human gene that encodes for the enzyme heme oxygenase 1, has been found to be a key component. The role of HMOX1 is to metabolize heme into bilirubin, carbon monoxide, and free iron by a two-step process. The first and rate-limiting step is the production of biliverdin and carbon monoxide from heme by HMOX1. The second step is the production of bilirubin from biliverdin by biliverdin reductase. Both bilirubin and carbon monoxide have been shown to scavenge ROS and to have potent anti-oxidant and anti-inflammatory activities. Agents that induce production of HMOX1 have been shown to have beneficial activity in models of diabetes, cardiovascular disease, hypertension, and pulmonary function.

Compound (I), 2-[(1,3-benzoxazol-2-yl)amino]-N-[2-(2-hydroxyethoxy)ethyl]-1-methyl-1H-benzimidazole-5-carboxamide (also referred to as: 2-(benzo[d]oxazol-2-ylamino)-N-(2-(2-hydroxyethoxy)ethyl)-1-methyl-1H-benzo[d]imidazole-5-carboxamide) whose structure is shown below, is an HMOX1 inducer disclosed in WO2020/210339, the

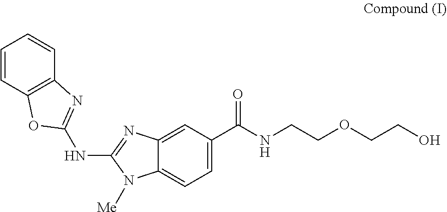 [SMILES: Cn1c(Nc2nc3ccccc3o2)nc2cc(C(=O)NCCOCCO)ccc21]

entire teachings of which are incorporated herein by reference. There is a need for salt forms of Compound (I) that are crystalline, low hygroscopic and otherwise have physical properties that are amenable to large scale manufacture; and also provide for good exposure in the patient after administration.

### SUMMARY OF THE INVENTION

It has been found that the phosphoric acid salt of Compound (I), i.e. 2-[(1,3-benzoxazol-2-yl)amino]-N-[2-(2-hydroxyethoxy)ethyl]-1-methyl-1H-benzimidazole-5-carboxamide monophosphate, can be crystallized under well-defined conditions to provide low hygroscopic crystalline forms. For the phosphoric acid salt of Compound (I), the molar ratio between Compound (I) and phosphoric acid is stoichiometrically 1:1. The 1:1 phosphoric acid salt of Compound (I) is referred to herein as “1:1 Compound (I) Phosphate”. The 1:1 molar ratio between phosphoric acid and Compound (I) refers to the whole number stoichiometric ratio between the phosphoric acid and Compound (I) in the salt. It is to be understood that the actual molar ratio between phosphoric acid and Compound (I) in a sample may vary slightly, such as, for example plus or minus 10%. 1:1 Compound (I) Phosphate has several advantageous properties when compared with the corresponding free form. For example, 1:1 Compound (I) phosphate shows roughly twice the exposure compared to the corresponding free form in Cynomolgus monkeys (10 and 100 mpk oral dose, see Example 6). These favorable properties make 1:1 Compound (I) Phosphate superior to the free form. Additionally, 1:1 Compound (I) phosphate shows low hygroscopicity compared with the corresponding citrate salt (see Example 4).

In one aspect, the present invention provides a phosphate salt of Compound (I) wherein the molar ratio between Compound (I) and phosphoric acid is stoichiometrically 1:1. As noted above, this salt is also referred to herein as “1:1 Compound (I) Phosphate”. Also included in the invention is a crystal form for 1:1 Compound (I) Phosphate, referred to herein as the “Compound (I) Phosphate Crystal Form”.

In another aspect, the present invention provides a novel crystal form of the free base the Compound (I) (hereinafter the “Compound (I) Crystal Form”).

In another aspect, the present invention provides a pharmaceutical composition comprising: i) 1:1 Compound (I) Phosphate, Compound (I) Phosphate Crystal Form or Compound (I) Crystal Form; and ii) a pharmaceutically acceptable carrier or diluent.

The present invention provides a method of increasing the activity of or the amount of HMOX-1 in a subject comprising administering to the subject an effective amount of 1:1 Compound (I) Phosphate, Compound (I) Phosphate Crystal Form, Compound (I) Crystal Form or the pharmaceutical composition comprising: i) 1:1 Compound (I) Phosphate, Compound (I) Phosphate Crystal Form or Compound (I) Crystal Form; and ii) a pharmaceutically acceptable carrier or diluent.

The present invention also provides a method of activating transcription factor Nrf2 in a subject comprising administering to the subject an effective amount of 1:1 Compound (I) Phosphate, Compound (I) Phosphate Crystal Form, Compound (I) Crystal Form or the pharmaceutical composition comprising: i) 1:1 Compound (I) Phosphate, Compound (I) Phosphate Crystal Form or Compound (I) Crystal Form; and ii) a pharmaceutically acceptable carrier or diluent.

The present invention also provides a method of reducing the amount of ROS in a subject comprising administering to the subject an effective amount of 1:1 Compound (I) Phosphate, Compound (I) Phosphate Crystal Form, Compound (I) Crystal Form or the pharmaceutical composition comprising: i) 1:1 Compound (I) Phosphate, Compound (I) Phosphate Crystal Form or the Compound (I) Crystal Form; and ii) a pharmaceutically acceptable carrier or diluent.

The present invention also provides an effective amount of 1:1 Compound (I) Phosphate, Compound (I) Phosphate Crystal Form, Compound (I) Crystal Form or the pharmaceutical composition comprising: i) 1:1 Compound (I) Phosphate, Compound (I) Phosphate Crystal Form or Compound (I) Crystal Form; and ii) a pharmaceutically acceptable carrier or diluent for use in increasing the activity of HMOX-1 in a subject, activating transcription factor Nrf2 in a subject or reducing the amount of ROS in a subject.

The present invention also provides the use of an effective amount of 1:1 Compound (I) Phosphate, Compound (I) Phosphate Crystal Form, Compound (I) Crystal Form or the pharmaceutical composition comprising: i) 1:1 Compound (I) Phosphate, Compound (I) Phosphate Crystal Form or Compound (I) Crystal Form; and ii) a pharmaceutically acceptable carrier or diluent in the manufacture of a medicament for increasing the activity of HMOX-1 in a subject, activating transcription factor Nrf2 in a subject or reducing the amount of ROS in a subject.

The present invention also provides the use of an effective amount of 1:1 Compound (I) Phosphate, Compound (I) Phosphate Crystal Form, Compound (I) Crystal Form or the pharmaceutical composition comprising: i) 1:1 Compound (I) Phosphate, Compound (I) Phosphate Crystal Form or Compound (I) Crystal Form; and ii) a pharmaceutically acceptable carrier or diluent for increasing the activity of HMOX-1 in a subject, activating transcription factor Nrf2 in a subject or reducing the amount of ROS in a subject.

### BRIEF DESCRIPTION OF THE DRAWINGS

FIG. 1 shows the X-ray Powder Diffraction (XRPD) pattern for Compound (I) Phosphate Crystal Form of Example 2-1.

FIG. 2 shows the Differential Scanning Calorimetry Analysis (DSC) thermogram for Compound (I) Phosphate Crystal Form of Example 2-1.

FIG. 3 shows the XRPD pattern for a lower crystallinity form due to disorder (lattice defect) of Compound (I) Phosphate Crystal Form of Example 2-2.

FIG. 4 shows the DSC thermogram for a lower crystallinity form due to disorder (lattice defect) of Compound (I) Phosphate Crystal Form of Example 2-2.

FIG. 5 shows the XRPD pattern for a lower crystallinity form due to disorder (lattice defect) of Compound (I) Phosphate Crystal Form of Example 2-3.

FIG. 6 shows the DSC thermogram for a lower crystallinity form due to disorder (lattice defect) of Compound (I) Phosphate Crystal Form of Example 2-3.

FIG. 7 shows the XRPD pattern for a lower crystallinity form due to disorder (lattice defect) of Compound (I) Phosphate Crystal Form of Example 2-4.

FIG. 8 shows the DSC thermogram for a lower crystallinity form due to disorder (lattice defect) of Compound (I) Phosphate Crystal Form of Example 2-4.

FIG. 9 shows the XRPD pattern for Compound (I) Crystal Form of Example 3.

FIG. 10 shows the DSC thermogram for Compound (I) Crystal Form of Example 3.

### DETAILED DESCRIPTION OF THE INVENTION

The present invention provides 1:1 Compound (I) Phosphate, Compound (I) Phosphate Crystal Form, Compound (I) Crystal Form, and their corresponding pharmaceutical compositions. The present invention also provides methods for increasing the activity of HMOX-1 in a human subject, for activating transcription factor Nrf2 in a human subject or for reducing the amount of ROS in a human subject by administering an effective amount of 1:1 Compound (I) Phosphate, Compound (I) Phosphate Crystal Form, Compound (I) Crystal Form or their corresponding pharmaceutical composition to the subject.

### Crystal Forms of 1:1 Compound (I) Phosphate and Compound (I)

In a particular embodiment, at least a particular percentage by weight of 1:1 Compound (I) Phosphate or Compound (I) is crystalline. Particular weight percentages include 70%, 72%, 75%, 77%, 80%, 82%, 85%, 86%, 87%, 88%, 89%, 90%, 91%, 92%, 93%, 94%, 95%, 96%, 97%, 98%, 99%, 99.5%, 99.9%, or a weight percentage of 70%-75%, 75%-80%, 80%-85%, 85%-90%, 90%-95%, 95%-100%, 70-80%, 80-90%, 90-100%. For example, in one embodiment, at least 90% (e.g., at least 95% or 99%) by weight of 1:1 Compound (I) Phosphate or Compound (I) is crystalline. It is to be understood that all values and ranges between these values and ranges are meant to be encompassed by the present invention.

In another particular embodiment, at least a particular percentage by weight of 1:1 Compound (I) Phosphate or Compound (I) is in a single crystal form. Particular weight percentages include 70%, 72%, 75%, 77%, 80%, 82%, 85%, 86%, 87%, 88%, 89%, 90%, 91%, 92%, 93%, 94%, 95%, 96%, 97%, 98%, 99%, 99.5%, 99.9%, or a weight percentage of 70%-75%, 75%-80%, 80%-85%, 85%-90%, 90%-95%, 95%-100%, 70-80%, 80-90%, 90-100%. For example, in one embodiment, at least 90% (e.g., at least 95% or 99%) by weight of the 1:1 Compound (I) Phosphate or Compound (I) is in a single crystal form. It is to be understood that all values and ranges between these values and ranges are meant to be encompassed by the present invention.

As used herein, “crystalline” refers to a solid having a crystal structure wherein the individual molecules have a highly homogeneous regular locked-in chemical configuration. Compound (I) Phosphate Crystal Form and Compound (I) Crystal Form can be crystals of a single crystal form of 1:1 Compound (I) Phosphate and Compound (I), or a mixture of crystals of different single crystal forms. As used herein, “single crystal form” means Compound (I) Phosphate Crystal Form or Compound (I) Crystal Form as a single crystal or a plurality of crystals in which each crystal has the same crystal form including the disordered form as mentioned below.

When a particular percentage by weight of 1:1 Compound (I) Phosphate (or Compound (I)) is a single crystal form, the remainder of 1:1 Compound (I) Phosphate (or Compound (I)) is some combination of amorphous phosphate salt (or amorphous Compound (I)), and/or one or more other crystal forms of 1:1 Compound (I) Phosphate (or Compound (I)) excluding the single crystal form. When the crystalline 1:1 Compound (I) Phosphate (or Compound (I)) is defined as a specified percentage of one particular crystal form of 1:1 Compound (I) Phosphate (or Compound (I)), the remainder is made up of amorphous form and/or crystal forms other than the particular form that is specified. Examples of a single crystal form include 1:1 Compound (I) Phosphate characterized by one or more properties as discussed herein.

### Characterization of Crystalline Forms of 1:1 Compound (I) Phosphate and Compound (I)

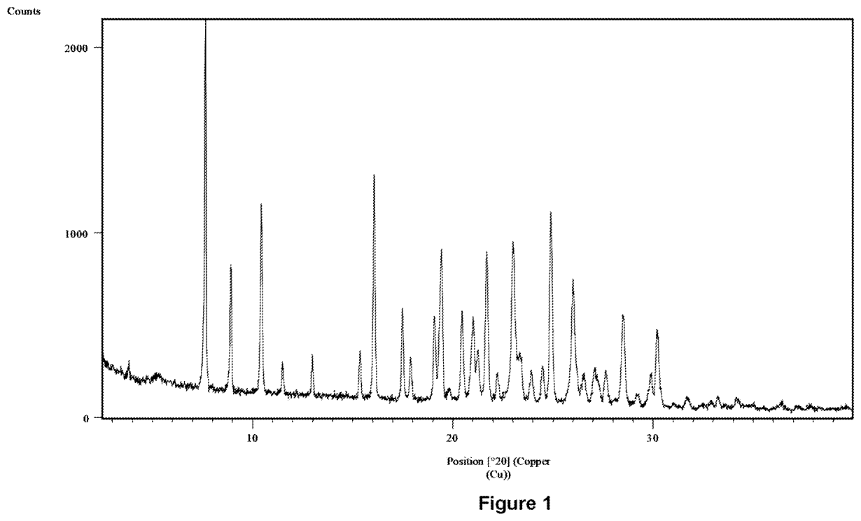

Samples are irradiated with copper K-alpha X-rays, wavelength, λ, of 1.5418 Å with the X-ray tube operated at 45 kV/40 mA; scanning 0.013 degrees/step. In one embodiment, Compound (I) Phosphate Crystal Form is a single crystal form. In a specific embodiment, Compound (I) Phosphate Crystal Form is characterized by the XRPD pattern shown in FIG. 1. In a more particular embodiment, Compound (I) Phosphate Crystal Form is characterized by an XRPD pattern which comprises characteristic peaks (20 angles±0.2°) at:
a) 7.7°, 10.4° and 16.10 in 20 (major peaks); or
b) 7.7°, 8.9°, 10.4°, 13.0° and 16.1° in 20; or
c) 7.7°, 8.9°, 10.4°, 11.5°, 13.0°, 15.4°, 16.1°, 17.5°, 17.9° and 21.7° in 20; or
d) 7.7°, 8.9°, 10.4°, 11.5°, 13.0°, 15.4°, 16.1°, 17.5°, 17.9°, 19.1°, 19.4°, 20.4°, 21.7°, 23.0° and 24.9° in 20.

A listing of XRPD peaks for Compound (I) Phosphate Crystal Form is found in Table 1 in Example 2-1. A specified 20 angle means the specified value±0.2°.

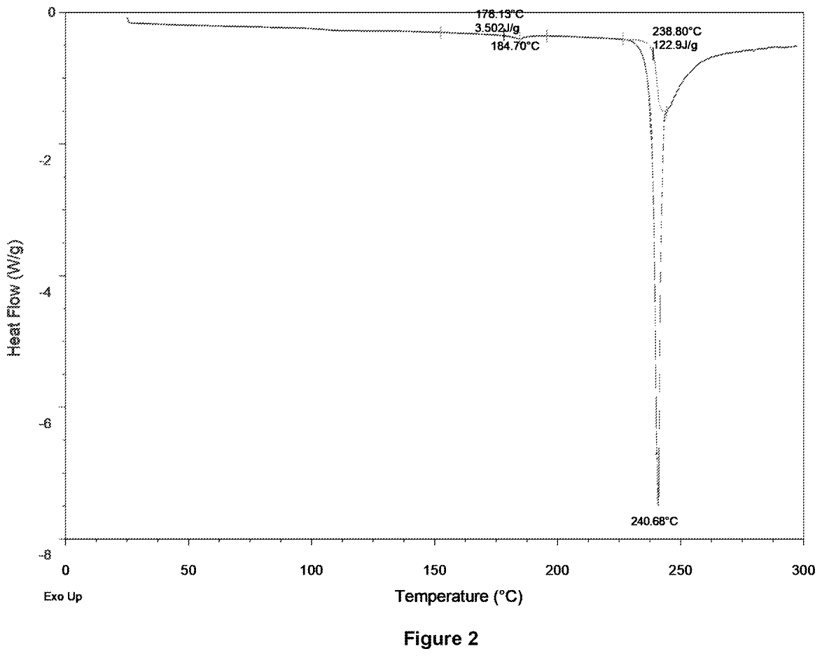

In another specific embodiment, Compound (I) Phosphate Crystal Form is characterized by a DSC phase transition onset temperature of 239° C. Alternatively, Compound (I) Phosphate Crystal Form is characterized by a DSC phase transition onset temperatures of 178° C. In another alternative, Compound (I) Phosphate Crystal Form is characterized by DSC phase transition onset temperatures of 178° C. and 239° C. The DSC for Compound (I) Phostphate Crystal Form is shown in FIG. 2. DSC phase transition onset temperatures are ±3° C. using a heating rate of 10° C. per minute.

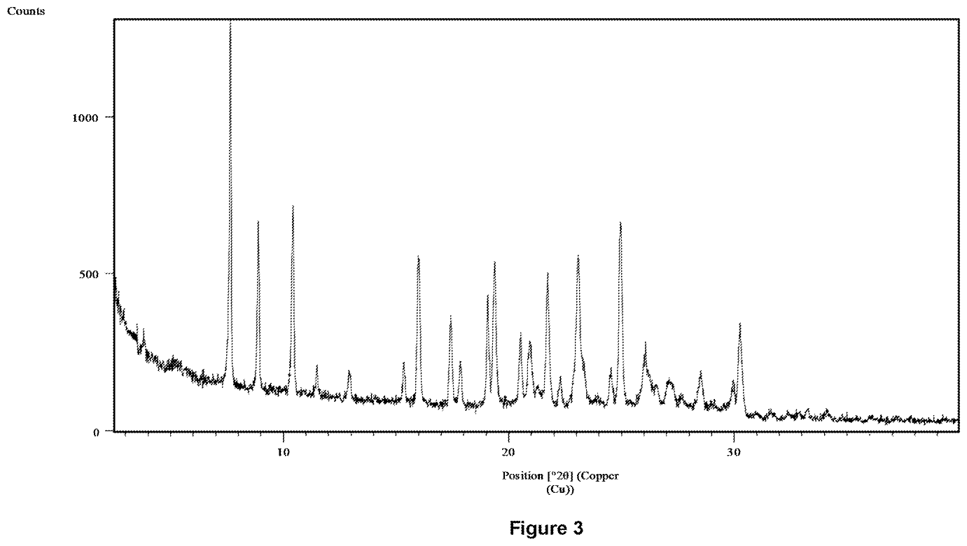

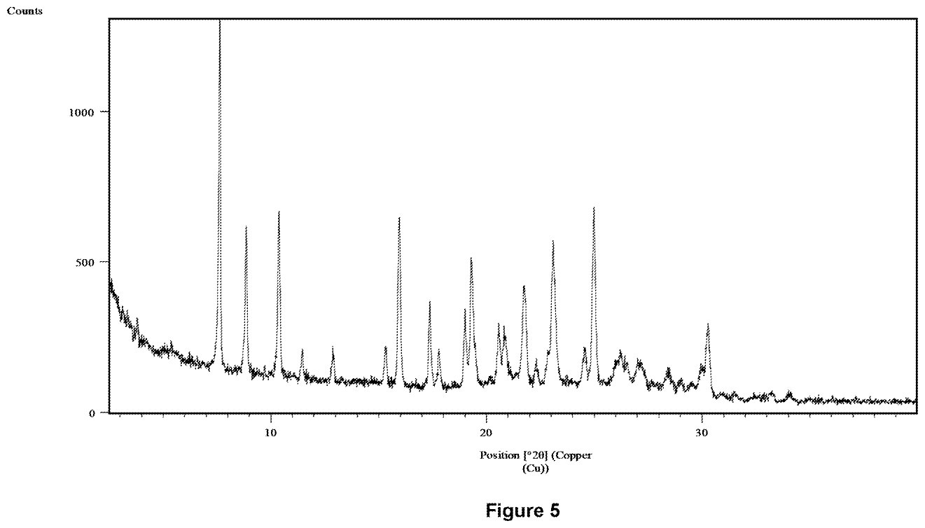

In another embodiment, Compound (I) Phosphate Crystal Form is characterized by lower crystallinity form due to disorder, also referred to as lattice defect. When a lattice defect is present, the regular and an ordered arrangement of atoms is disrupted such that structural disorder exists. The disordered form of Compound (I) Phosphate Crystal Form has almost the same characteristic XRPD peaks recited above for the ordered form within ±0.2°. Therefore, the characteristic peaks for Compound (I) Phosphate Crystal Form recited above are meant to comprise both the ordered and disordered forms. In a more specific embodiment, the ordered form of Compound (I) Phosphate Crystal Form additionally comprises XRPD peaks (2θangles±0.2°) at 21.2°, 23.9°, 26.0° and/or 28.5° in 2θ. FIGS. 3 and 5 show the XRPD pattern of the disordered form of Compound (I) Phosphate Crystal Form; and the peak listing for the disordered form of Compound (I) Phosphate Crystal Form is provided in Table 2 of Example 2-2, Table 3 of Example 2-3 and Table 4 of Example 2-4. A specified 20 angle means the specified value±0.2°.

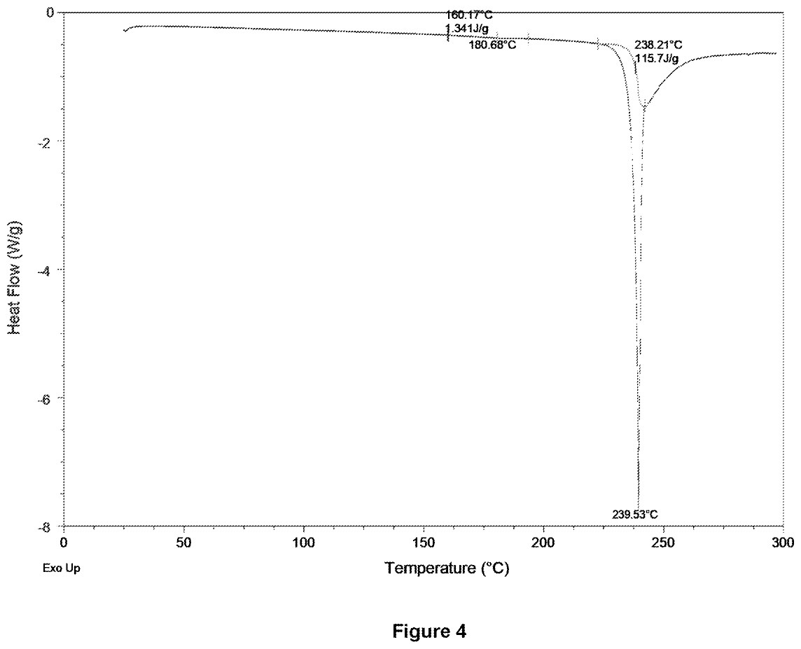

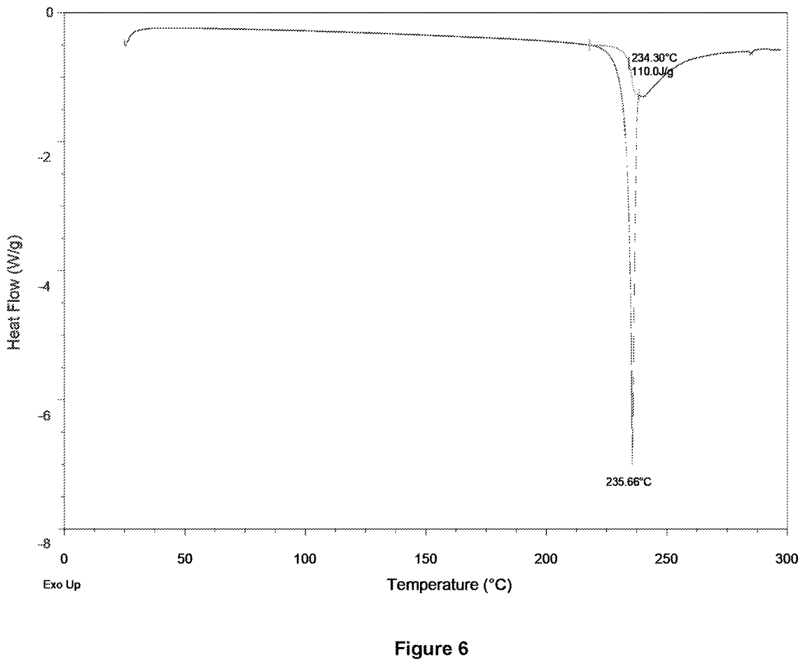

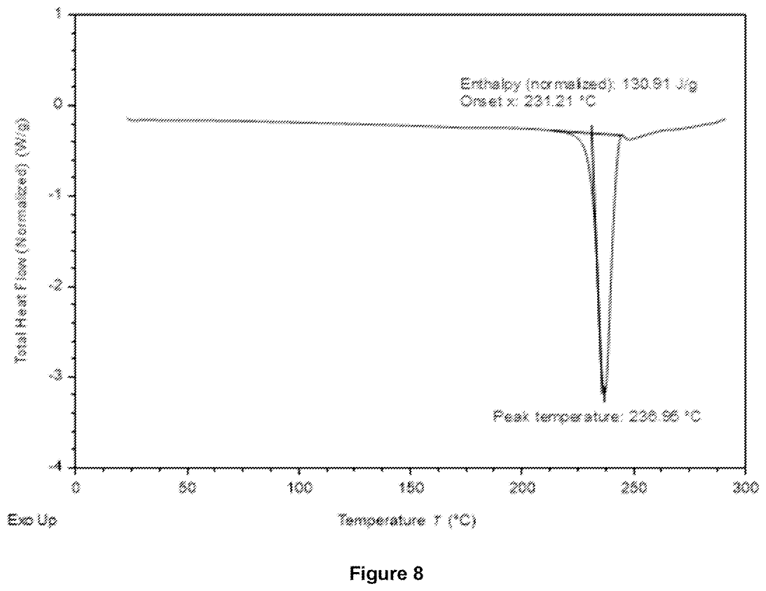

The degree of disorder, and, consequently, the crystallinity of the disordered form can vary. Without being bound by theory, it is believed that the degree of disorder varies according to the impurity level of Compound (I) and/or the solvent used in crystallization of 1:1 Compound (I) Phosphate, and that the degree of disorder is reflected by a lowering of the DSC phase transition onset temperature. In one embodiment, the DSC phase transition onset temperature is between 227° C. and 239° C. In another embodiment, the disordered form of Compound (I) Phosphate Crystal Form is characterized by DSC phase transition onset temperature of 238° C., as shown in FIG. 4, 234° C., as shown in FIG. 6, or 231° C., as shown in FIG. 8. DSC phase transition onset temperatures are ±3° C. using a heating rate of 10° C. per minute.

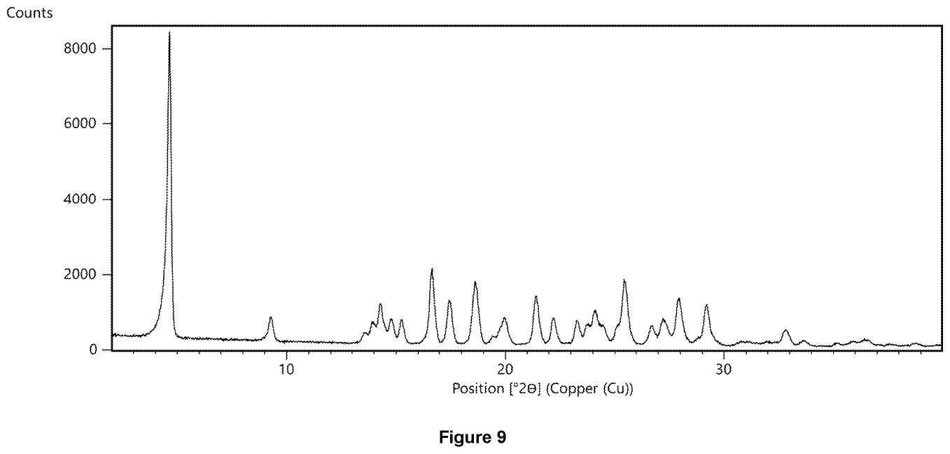

In another embodiment, Compound (I) Crystal Form is characterized by the XRPD pattern shown in FIG. 9. In a more particular embodiment, Compound (I) Crystal Form is characterized by an XRPD pattern which comprises peaks (2θangles±0.2°) at:
a) 4.6°, 16.6° and 18.6° in 2θ(major peaks); or
b) 4.6°, 14.3°, 16.6° and 18.6° in 20; or
c) 4.6°, 13.6°, 13.9°, 14.3°, 14.8°, 16.6°, 18.6°, 19.9° and 22.2° in 20; or
d) 4.6°, 9.2°, 13.6°, 13.9°, 14.3°, 14.8°, 15.2°, 16.6°, 17.4°, 18.6°, 19.4°, 19.9°, 21.4° and 22.2° in 20.

A listing of XRPD peaks for Compound (I) Crystal Form is found in Table 5 in Example 3. A specified 2θangle means the specified value±0.2°.

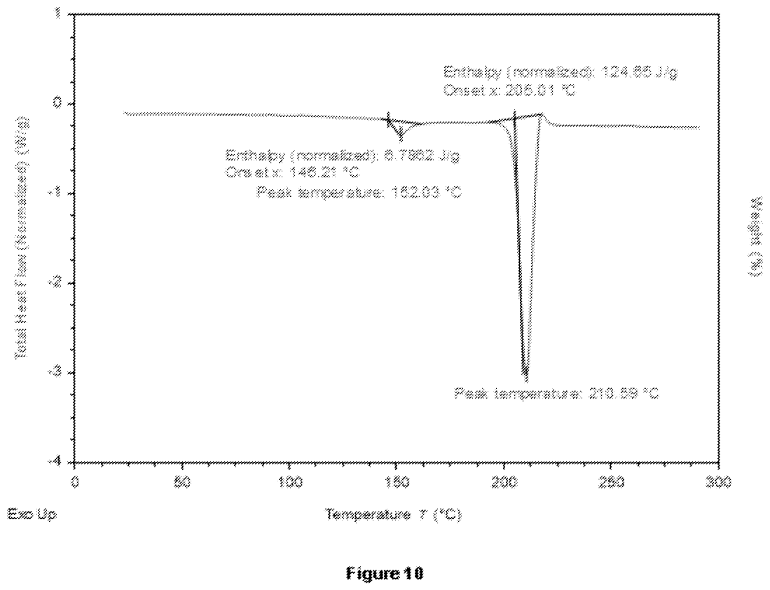

In another specific embodiment, Compound (I) Crystal Form is characterized by DSC phase transition onset temperatures of 205° C., as shown in FIG. 10. DSC phase transition onset temperatures are ±3° C. using a heating rate of 10° C. per minute.

### Methods of Treatment

In certain embodiments, the invention provides methods of increasing the activity of or the amount of HMOX1 in a human subject comprising: administering to a human subject an effective amount of the 1:1 Compound (I) Phosphate, Compound (I) Phosphate Crystal Form, Compound (I) Crystal Form, or an effective amount of a pharmaceutical composition comprising any of the foregoing.

In certain embodiments, the invention provides methods of activating transcription factor Nrf2 in a human subject comprising: administering to a human subject an effective amount of the 1:1 Compound (I) Phosphate, Compound (I) Phosphate Crystal Form, Compound (I) Crystal Form, or an effective amount of a pharmaceutical composition comprising any of the foregoing.

In certain embodiments, the invention provides methods of reducing the amount of ROS in a human subject comprising: administering to a human subject an effective amount of the 1:1 Compound (I) Phosphate, Compound (I) Phosphate Crystal Form, Compound (I) Crystal Form, or an effective amount of a pharmaceutical composition comprising any of the foregoing.

In certain embodiments, the invention provides methods for using an effective amount of the 1:1 Compound (I) Phosphate, Compound (I) Phosphate Crystal Form, Compound (I) Crystal Form, or an effective amount of a pharmaceutical composition comprising any of the foregoing. 1:1 Compound (I) Phosphate, Compound (I) Phosphate Crystal Form, Compound (I) Crystal Form, or a pharmaceutical composition comprising any of the foregoing may be useful for a variety of therapeutic applications including, for example, treating and/or reducing a wide variety of diseases and disorders including, for example, fibrotic diseases, neurodegenerative disease, cardiovascular disease, renal disease, inflammatory disease, liver disease, eye disease, thyroid disease, viral infection, osteoporosis, pregnancy disorders, endometriosis, diabetes, cancers, skin diseases, mitochondrial diseases, hematological disorders, and muscle diseases. The methods comprise administering to a subject in need thereof a pharmaceutically effective amount of one or more compounds of the invention, a pharmaceutically acceptable salt thereof, and/or pharmaceutical compositions thereof.

Compounds that increase levels or activity of HMOX1 are potentially useful in treating diseases or conditions that may be associated at least in part with oxidative stress such as, but not limited to, fibrotic diseases, neurodegenerative disease, cardiovascular disease, renal disease, inflammatory disease, liver disease, eye disease, thyroid disease, viral infection, osteoporosis, pregnancy disorders, endometriosis, diabetes, cancers, skin diseases, mitochondrial diseases, hematological disorders, and muscle diseases. As used herein, the diseases or conditions associated with oxidative stress also include chronic effects (e.g., tissue damage, chronic inflammation) associated with persistent or long-term increases in oxidative stress due to the diseases or conditions described herein.

Fibrotic diseases associated with oxidative stress include, but are not limited to, fibrotic diseases of the lung such as chronic obstructive pulmonary disease (COPD), idiopathic pulmonary fibrosis, and sarcoidosis; fibrotic diseases of the liver including those caused by alcoholic cirrhosis, steatosis, cholestasis, drug side effect, and viral infection; and fibrotic diseases of the skin including autoimmune diseases such as scleroderma and psoriasis.

Neurodegenerative diseases associated with oxidative stress include, but are not limited to, Friedreich's ataxia, Alzheimer's disease, Parkinson's disease, amyotrophic lateral sclerosis, multiple sclerosis, cerebral nerve degenerative disease, and Charcot-Marie-Tooth syndrome.

Cardiovascular diseases associated with oxidative stress include, but are not limited to, hypertension, heart failure, hypercholesterolaemia, atherosclerosis, arteriosclerosis, thrombosis, acute coronary thrombosis, deep vein thrombosis, peripheral vascular disease, congestive heart failure, acute coronary syndrome, failure of arterial fistula for dialysis, ischemia reperfusion injury, primary pulmonary hypertension, primary pulmonary arterial hypertension, and secondary pulmonary arterial hypertension.

Renal diseases associated with oxidative stress include, but are not limited to, acute kidney injury, polycystic kidney disease, Alport syndrome, diabetic nephropathy, glomerular nephritis, lupus nephritis, sickle cell nephropathy, and acute tubular necrosis.

Inflammatory diseases associated with oxidative stress include, but are not limited to, asthma, chronic obstructive pulmonary disease, idiopathic pulmonary fibrosis, inflammatory bowel syndrome, Crohn's disease, celiac disease, ulcerative colitis, chronic inflammatory bowel disease, scleroderma, dermatitis, systemic lupus erythematosus, esophagitis, vasculitis, pancreatitis, tendonitis, osteoarthritis, rheumatoid arthritis, ankylosing spondylitis, and chronic inflammation of the brain.

Liver diseases associated with oxidative stress include, but are not limited to, drug induced liver toxicity, nonalcoholic steatohepatitis, and hepatitis, e.g., hepatitis B infection and hepatitis C infection.

Eye diseases and conditions associated with oxidative stress include, but are not limited to, conjunctivitis, glaucoma, uveitis, wound healing (e.g., after surgery such as LASIK), eye trauma, corneal grafts, Fuchs' endothelial corneal dystrophy, macular degeneration, cataracts, light retinopathy, retinitis pigmentosa, diabetic retinopathy, and retinopathy of prematurity, as well as inflammation and tissue damage associated with these diseases.

Thyroid diseases associated with oxidative stress include, but are not limited to, Graves' disease, follicular adenoma, and papillary and follicular carcinomas.

Lung diseases associated with oxidative stress include, but are not limited to, bronchitis, asthma, chronic obstructive pulmonary disease, idiopathic pulmonary fibrosis, pulmonary bronchitis, bronchiectasis, pulmonary edema, and emphysema.

Skin diseases associated with oxidative stress include, but are not limited to, dermatitis, scleroderma, and psoriasis.

Viral infections associated with oxidative stress include both viral replication of viruses, as well as tissue damage (e.g., fibrosis) due to oxidative stress resulting from chronic viral infection, for viruses including but are not limited to human immunodeficiency virus, hepatitis B, hepatitis C, and herpesvirus.

Diabetic conditions include, but are not limited to, type 1 diabetes mellitus, type 2 diabetes mellitus, gestational diabetes, pre-diabetes, hyperglycemia, and metabolic syndrome as well as secondary conditions resulting from diabetic conditions (e.g., congestive heart failure and nephropathy).

Mitochondrial disease associated with oxidative stress include, but are not limited to, mitochondrial myopathies, Leber's hereditary optic neuropathy (LHON), myoclonic epilepsy with ragged red fibers (MERFF), mitochondrial encephalomyopathy, lactic acidosis and stroke-like episodes (MELAS) or Leigh's Syndrome.

Hematological disorders associated with oxidative stress include, but are not limited to, Diamond Blackfan anemia, myelodysplastic syndrome, sickle cell disease and beta-thalessemia.

Muscle diseases associated with oxidative stress include, but are not limited to, Duchenne muscular dystrophy, limb girdle muscular dystrophy, Becker muscular dystrophy, myotonic dystrophy and rhabdomyolysis.

Cancers associated with oxidative stress include, but are not limited to, breast cancer, colorectal cancer, lung cancer, ovarian cancer, uterine cancer, prostate cancer, leukemias, lymphomas, brain cancer (including glioblastoma multiforme and neuroblastoma), head and neck cancer, pancreatic cancer, melanoma, hepatocellular carcinoma, renal cancer, and soft tissue sarcomas. In one embodiment, the cancer is breast cancer, colon cancer, and ovarian cancer. In one embodiment, the cancer is selected from leukemia, acute myeloid leukemia, chronic myelogenous leukemia, breast cancer, brain cancer, colon cancer, colorectal cancer, head and neck cancer, hepatocellular carcinoma, lung adenocarcinoma, metastatic melanoma, pancreatic cancer, prostate cancer, ovarian cancer and renal cancer. In one embodiment, the cancer is lung cancer, colon cancer, brain cancer, neuroblastoma, prostate cancer, melanoma, glioblastoma multiforme or ovarian cancer. In another embodiment, the cancer is lung cancer, breast cancer, colon cancer, brain cancer, neuroblastoma, prostate cancer, melanoma, glioblastoma multiforme or ovarian cancer. In yet another embodiment, the cancer is breast cancer, colon cancer and lung cancer. In another embodiment, the cancer is a breast cancer. In yet another embodiment, the cancer is a basal sub-type breast cancer or a luminal B sub-type breast cancer. In yet another embodiment, the cancer is a basal sub-type breast cancer. In yet another embodiment, the basal sub-type breast cancer is ER (estrogen receptor), HER2 and PR (progesterone receptor) negative breast cancer. In yet another embodiment, the cancer is a soft tissue cancer. A “soft tissue cancer” is an art-recognized term that encompasses tumors derived from any soft tissue of the body. Such soft tissue connects, supports, or surrounds various structures and organs of the body, including, but not limited to, smooth muscle, skeletal muscle, tendons, fibrous tissues, fatty tissue, blood and lymph vessels, perivascular tissue, nerves, mesenchymal cells and synovial tissues. Thus, soft tissue cancers can be of fat tissue, muscle tissue, nerve tissue, joint tissue, blood vessels, lymph vessels, and fibrous tissues. Soft tissue cancers can be benign or malignant. Generally, malignant soft tissue cancers are referred to as sarcomas, or soft tissue sarcomas. There are many types of soft tissue tumors, including lipoma, lipoblastoma, hibernoma, liposarcoma, leiomyoma, leiomyosarcoma, rhabdomyoma, rhabdomyosarcoma, neurofibroma, schwannoma (neurilemoma), neuroma, malignant schwannoma, neurofibrosarcoma, neurogenic sarcoma, nodular tenosynovitis, synovial sarcoma, hemangioma, glomus tumor, hemangiopericytoma, hemangioendothelioma, angiosarcoma, Kaposi sarcoma, lymphangioma, fibroma, elastofibroma, superficial fibromatosis, fibrous histiocytoma, fibrosarcoma, fibromatosis, dermatofibrosarcoma protuberans (DFSP), malignant fibrous histiocytoma (MFH), myxoma, granular cell tumor, malignant mesenchymomas, alveolar soft-part sarcoma, epithelioid sarcoma, clear cell sarcoma, and desmoplastic small cell tumor. In a particular embodiment, the soft tissue cancer is a sarcoma selected from the group consisting of a fibrosarcoma, a gastrointestinal sarcoma, a leiomyosarcoma, a dedifferentiated liposarcoma, a pleomorphic liposarcoma, a malignant fibrous histiocytoma, a round cell sarcoma, and a synovial sarcoma.

Thus the present invention provides a method of treatment comprising administering to a subject the 1:1 Compound (I) Phosphate, Compound (I) Phosphate Crystal Form, Compound (I) Crystal Form, or a pharmaceutical composition comprising any of the foregoing so as to treat at least one of the diseases or conditions listed above.

A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

### Methods of Administration and Dosage Forms

The precise amount of compound administered to provide an “effective amount” to the subject will depend on the mode of administration, the type, and severity of the disease or condition, and on the characteristics of the subject, such as general health, age, sex, body weight, and tolerance to drugs. The skilled artisan will be able to determine appropriate dosages depending on these and other factors. When administered in combination with other therapeutic agents, e.g., when administered in combination with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of the invention being used by following, for example, dosages reported in the literature and recommended in the Physician's Desk Reference (57th Ed., 2003).

The term “effective amount” means an amount when administered to the subject which results in beneficial or desired results, including clinical results, e.g., inhibits, suppresses or reduces the symptoms of the condition being treated in the subject as compared to a control. For example, a therapeutically effective amount can be given in unit dosage form (e.g., 0.1 mg to about 50 g per day, alternatively from 1 mg to about 5 grams per day; and in another alternatively from 10 mg to 1 gram per day).

The terms “administer”, “administering”, “administration”, and the like, as used herein, refer to methods that may be used to enable delivery of compositions to the desired site of biological action. These methods include, but are not limited to, intraarticular (in the joints), intravenous, intramuscular, intratumoral, intradermal, intraperitoneal, subcutaneous, oral, topical, intrathecal, inhalational, transdermal, rectal, and the like. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa.

In addition, the 1:1 Compound (I) Phosphate, Compound (I) Phosphate Crystal Form, Compound (I) Crystal Form, or a pharmaceutical composition can be co-administered with other therapeutic agents. As used herein, the terms “co-administration”, “administered in combination with”, and their grammatical equivalents, are meant to encompass administration of two or more therapeutic agents to a single subject, and are intended to include treatment regimens in which the agents are administered by the same or different route of administration and at the same or different times. In some embodiments the compounds described herein will be co-administered with other agents. These terms encompass administration of two or more agents to the subject so that both agents and/or their metabolites are present in the subject at the same time. They include simultaneous administration in separate compositions, administration at different times in separate compositions, and/or administration in a composition in which both agents are present. Thus, in some embodiments, the compounds described herein and the other agent(s) are administered in a single composition. In some embodiments, the compounds described herein and the other agent(s) are admixed in the composition.

The particular mode of administration and the dosage regimen will be selected by the attending clinician, taking into account the particulars of the case (e.g. the subject, the disease, the disease state involved, the particular treatment). Treatment can involve daily or multi-daily or less than daily (such as weekly or monthly etc.) doses over a period of a few days to months, or even years. However, a person of ordinary skill in the art would immediately recognize appropriate and/or equivalent doses looking at dosages of approved compositions for treating a disease using the 1:1 Compound (I) Phosphate, Compound (I) Phosphate Crystal Form, Compound (I) Crystal Form, or a pharmaceutical composition for guidance.

### Pharmaceutical Compositions Including 1:1 Compound (I) Phosphate, Compound (I) Phosphate Crystal Form, and Compound (I) Crystal Form

1:1 Compound (I) Phosphate, Compound (I) Phosphate Crystal Form, or Compound (I) Crystal Form disclosed herein can be suitably formulated into pharmaceutical compositions for administration to a subject.

The pharmaceutical compositions of the present teachings optionally include one or more pharmaceutically acceptable carriers and/or diluents therefor, such as lactose, starch, cellulose and dextrose. Other excipients, such as flavoring agents, sweeteners; and preservatives, such as methyl, ethyl, propyl and butyl parabens, can also be included. More complete listings of suitable excipients can be found in the Handbook of Pharmaceutical Excipients (5^{th} Ed., Pharmaceutical Press (2005)). A person skilled in the art would know how to prepare formulations suitable for various types of administration routes.

Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. The carriers, diluents and/or excipients are “acceptable” in the sense of being compatible with the other ingredients of the pharmaceutical composition and not deleterious to the recipient thereof.

Typically, for oral therapeutic administration, a compound of the present teachings may be incorporated with excipient and used in the form of ingestible tablets, buccal tablets, troches, capsules, elixirs, suspensions, syrups, wafers, and the like.

Typically for parenteral administration, solutions of a compound of the present teachings can generally be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms.

Typically, for injectable use, sterile aqueous solutions or dispersion of, and sterile powders of, a compound described herein for the extemporaneous preparation of sterile injectable solutions or dispersions are appropriate.

For nasal administration, the compounds of the present teachings can be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. The aerosol dosage forms can also take the form of a pump-atomizer.

For buccal or sublingual administration, the compounds of the present teachings can be formulated with a carrier such as sugar, acacia, tragacanth, gelatin or glycerine, as tablets, lozenges or pastilles.

For rectal administration, the compounds described herein can be formulated in the form of suppositories containing a conventional suppository base such as cocoa butter.

The invention is illustrated by the following examples, which are not intended to be limiting in any way.

### EXPERIMENTAL

### Instruments and Methods

### X-ray Powder Diffraction (XRPD)

### Condition A:

Instrument: Panalytical Empyrean Powder Diffractometer (3^{rd} Generation)

Samples were irradiated at 25° C., with copper K-alpha X-rays with the X-ray tube operated at 45 kV/4θmA; scanning 0.013 degrees/step; Start Position 2.5006° 2θ; End Position 39.9856° 2θ.

### Condition B:

Instrument: Malvern Panalytical Empyrean Powder Diffractometer.

Samples were irradiated at 25° C., with copper K-alpha X-rays, wavelength, λ, of 1.5418 Å with the X-ray tube operated at 45 kV/4θmA; scanning 0.013 degrees/step; Start Position 2.5° 2θ; End Position 40.0° 2θ.

### Differential Scanning Calorimetry (DSC)

### Condition A:

Differential scanning calorimetry was performed with a TA Discovery series DSC using approximately a few milligrams of material in a Tzero aluminum pan sealed with a Tzero hermetic lid. Samples were analyzed using a heating rate of 10° C. per minute under 50 mL per minute of nitrogen flow.

### Condition B:

Differential scanning calorimetry was performed with a DSC Q2000 (TA Instruments) using a few milligrams of material in an aluminum pan without a lid. Samples were analyzed using a heating rate of 10° C. per minute under 50 mL per minute of nitrogen flow.

### Example 1: Salt Screening

Based on the pKa value (approximately 2.75, basic) and approximate solubility of the freebase form of Compound (I), a total of 65 salt and co-crystal screening experiments were conducted using 13 acids and five solvent systems. Specifically, about 20 mg Compound (I) and the corresponding acid were mixed in 0.5-0.75 mL solvents, with a molar charge ratio of 1:1 (acid/base). After magnetically stirring for 23 hours at room temperature, any solid was isolated by centrifugation. If there was no precipitation, the clear solutions were further stirred at 10° C. followed by cooling at 5° C. to induce crystallization. If still not solid, the final clear solutions were subjected to slow evaporation at room temperature. Any isolated solid were vacuum dried at 50° C. for 2 hours before analysis.

A total of thirteen crystalline salts were obtained. The phosphate, and citrate salts were selected for further evaluation in aqueous, fasted state simulated intestinal fluid (FaSSIF), fed state simulated intestinal fluid (FeSSIF) and simulated gastric fluid (SGF). Both the phosphate and citrate salts showed improved solubility in FaSSIF and SGF, compared to Compound (I). The phosphate salt showed lower hygroscopicity than the citrate salt and was further evaluated for PK in Cynomolgus macaques. After 10 and 100 mpk oral dose it showed 1.5 times higher exposure compare to free form Compound (I).

### Example 2: Preparation of Compound (I) Phosphate Crystal Form

### Example 2-1

To a stirred solution of 85% phosphoric acid (0.2 ml) in methanol (20 ml) was added Compound (I) (99.9% area purity by HPLC; 1.00 g) with methanol (5 ml) at room temperature under N₂ atmosphere. The mixture was stirred at room temperature for 2 days and then at 50° C. for 6 hours. To the mixture was added 85% phosphoric acid (51.8 μl) at 50° C., and the mixture was stirred at the same temperature for overnight, and then at room temperature for 2.5 hours. The precipitate was collected by filtration and washed with methanol (10 ml). The wet residue was dried at 50° C. for 5 hours under the reduced pressure to afford Compound (I) Phosphate Crystal Form (1.08 g) with 99.9% area purity by HPLC. The Compound (I) Phosphate Crystal Form thus obtained was characterized by XRPD (Condition B: FIG. 1) and DSC (Condition B: FIG. 2). It showed high crystallinity by XRPD, and a peak listing for the XRPD is provided below in Table 1.

TABLE 1

| Pos. [°2θ] | Height [cts] | Rel. Int. [%] |
|---|---|---|
| 3.8147 | 59.70 | 3.01 |
| 7.6558 | 1986.67 | 100.00 |
| 8.9128 | 657.67 | 33.10 |
| 10.4166 | 954.92 | 48.07 |
| 11.4873 | 162.30 | 8.17 |
| 12.9806 | 190.74 | 9.60 |
| 15.3607 | 244.87 | 12.33 |
| 16.0541 | 1205.58 | 60.68 |
| 17.4600 | 474.69 | 23.89 |
| 17.8671 | 223.93 | 11.27 |
| 19.0503 | 438.22 | 22.06 |
| 19.4396 | 729.72 | 36.73 |
| 20.4426 | 482.35 | 24.28 |
| 21.0101 | 426.79 | 21.48 |
| 21.2433 | 265.62 | 13.37 |
| 21.6920 | 792.35 | 39.88 |
| 22.1925 | 149.28 | 7.51 |
| 22.9685 | 825.14 | 41.53 |
| 23.4025 | 234.29 | 11.79 |
| 23.9076 | 165.02 | 8.31 |
| 24.4508 | 196.26 | 9.88 |
| 24.8800 | 1031.04 | 51.90 |
| 25.9694 | 653.93 | 32.92 |
| 26.5148 | 138.26 | 6.96 |
| 27.0469 | 183.39 | 9.23 |
| 27.6249 | 166.60 | 8.39 |
| 28.5052 | 473.65 | 23.84 |
| 29.2374 | 53.75 | 2.71 |
| 29.8746 | 166.88 | 8.40 |
| 30.1432 | 366.98 | 18.47 |
| 31.6976 | 43.55 | 2.19 |
| 33.2029 | 57.58 | 2.90 |
| 34.1684 | 44.97 | 2.26 |
| 36.3842 | 26.46 | 1.33 |
| 37.1776 | 10.63 | 0.54 |

### Example 2-2

To a stirred suspension of Compound (I) (99.6% area purity by HPLC; 500 mg) in a mixture of ethanol (16 ml) and water (4 ml) was added 85% phosphoric acid (0.13 ml) at room temperature, and the mixture was stirred at 90° C. After getting clear solution, it was stirred at 50° C. for 1 hour and then at room temperature for overnight. The precipitate was collected by filtration and washed with a mixture of ethanol (2 ml) and water (0.5 ml), and then ethanol (2.5 ml). The wet residue was dried at 50° C. for overnight under the reduced pressure to afford Compound (I) Phosphate Crystal Form (518 mg) with 99.8% area purity by HPLC. The Compound (I) Phosphate Crystal Form thus obtained was characterized by XRPD (Condition B: FIG. 3) and DSC (Condition B: FIG. 4). It showed lower crystallinity by XRPD, and a peak listing for the XRPD is provided below in Table 2.

TABLE 2

| Pos. [°2θ] | Height [cts] | Rel. Int. [%] |
|---|---|---|
| 7.6490 | 1124.02 | 100.00 |
| 8.8857 | 541.22 | 48.15 |
| 10.4151 | 598.90 | 53.28 |
| 11.4792 | 88.08 | 7.84 |
| 12.9294 | 74.12 | 6.59 |
| 15.3419 | 115.09 | 10.24 |
| 15.9632 | 448.74 | 39.92 |
| 17.4312 | 249.53 | 22.20 |
| 17.8342 | 137.89 | 12.27 |
| 19.0440 | 337.01 | 29.98 |
| 19.3786 | 452.84 | 40.29 |
| 20.5138 | 239.73 | 21.33 |
| 20.9397 | 199.17 | 17.72 |
| 21.7083 | 431.74 | 38.41 |
| 22.2804 | 91.91 | 8.18 |
| 23.0641 | 493.12 | 43.87 |
| 24.5084 | 124.47 | 11.07 |
| 24.9639 | 583.84 | 51.94 |
| 26.0285 | 188.59 | 16.78 |
| 26.5554 | 84.39 | 7.51 |
| 27.0563 | 99.36 | 8.84 |
| 28.5039 | 128.99 | 11.48 |
| 29.9216 | 95.90 | 8.53 |
| 30.2634 | 283.19 | 25.19 |
| 31.5885 | 14.98 | 1.33 |
| 33.2302 | 28.79 | 2.56 |
| 34.1166 | 22.78 | 2.03 |

### Example 2-3

To a stirred suspension of Compound (I) (96.7% area purity by HPLC; 2.00 g) in a mixture of ethanol (64 ml) and water (16 ml) was added 85% phosphoric acid (0.519 ml) at room temperature, and the mixture was stirred at 90° C. After getting clear solution, it was stirred at 60° C. for 1 hour and at 50° C. for overnight to obtain a slurry. The slurry was stirred at room temperature for overnight. The precipitate was collected by filtration and washed with a mixture of ethanol (8 ml) and water (2 ml), and then ethanol (10 ml). The wet residue was dried at 50° C. for 3 days under the reduced pressure to afford Compound (I) Phosphate Crystal Form (2.00 g) with 96.9% area purity by HPLC. The Compound (I) Phosphate Crystal Form thus obtained was characterized by XRPD (Condition B: FIG. 5) and DSC (Condition B: FIG. 6). It showed lower crystallinity by XRPD, and a peak listing for the XRPD is provided below in Table 3.

TABLE 3

| Pos. [°2θ] | Height [cts] | Rel. Int. [%] |
|---|---|---|
| 7.6371 | 1187.32 | 100.00 |
| 8.8594 | 491.45 | 41.39 |
| 10.3858 | 549.16 | 46.25 |
| 11.4654 | 86.27 | 7.27 |
| 12.9028 | 87.70 | 7.39 |
| 15.2957 | 116.71 | 9.83 |
| 15.9681 | 555.12 | 46.75 |
| 17.3643 | 286.17 | 24.10 |
| 17.7945 | 121.00 | 10.19 |
| 19.0055 | 264.31 | 22.26 |
| 19.2754 | 376.94 | 31.75 |
| 20.5718 | 197.36 | 16.62 |
| 20.8480 | 177.77 | 14.97 |
| 21.7389 | 339.33 | 28.58 |
| 22.3013 | 91.25 | 7.69 |
| 23.0940 | 497.74 | 41.92 |
| 24.5894 | 140.97 | 11.87 |
| 24.9878 | 588.87 | 49.60 |
| 26.2245 | 137.33 | 11.57 |
| 27.0372 | 106.59 | 8.98 |
| 28.4379 | 81.11 | 6.83 |
| 29.0127 | 55.28 | 4.66 |
| 30.2606 | 233.24 | 19.64 |
| 33.2430 | 23.66 | 1.99 |
| 34.0859 | 18.88 | 1.59 |

### Example 2-4

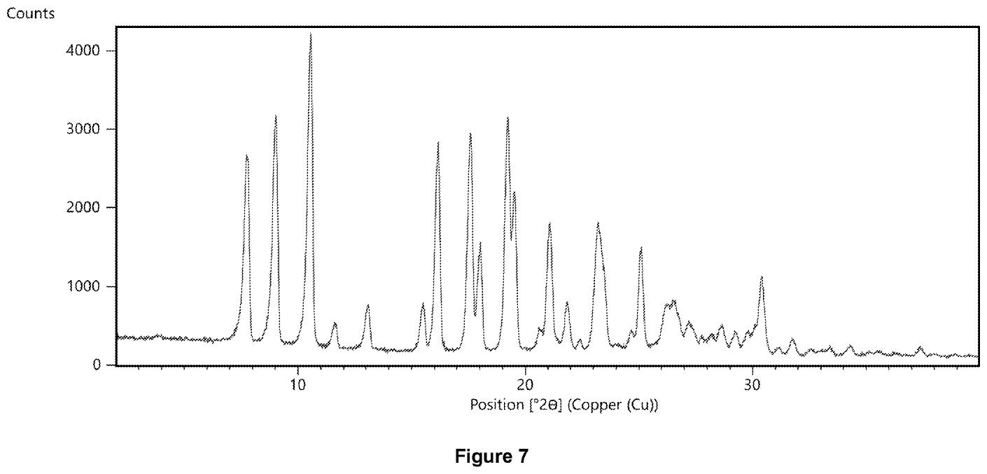

2.8 mL 85% Phosphoric acid was added to 350 mL methanol. To this solution 14 gm of compound (I) was gradually added. 300 mg of Compound (I) phosphate was added as seeds, and the resulting mixture was stirred for 48 hr. Additional 1.8 mL 85% Phosphoric acid was added to the reaction mixture and stirring was continued for 2 more days at 60-70° C. The resulting slurry was cooled to room temperature and filtered to isolate solid, which was dried under vacuum at 40° C. overnight. Yield 15.14 g. The Compound (I) Phosphate Crystal Form thus obtained was characterized by XRPD (Condition A: FIG. 7) and DSC (Condition A: FIG. 8). XRPD peak listing is provided below in Table 4.

TABLE 4

| Pos. [°2θ] | Height [cts] | Rel. Int. [%] |
|---|---|---|
| 7.6 | 2409.93 | 60.02 |
| 9 | 2925.16 | 72.85 |
| 10.5 | 4015.13 | 100.00 |
| 11.6 | 293.29 | 7.30 |
| 13.1 | 568.45 | 14.16 |
| 15.5 | 595.61 | 14.83 |
| 16.1 | 2675.48 | 66.63 |
| 17.6 | 2814.98 | 70.11 |
| 18 | 1348.84 | 33.59 |
| 19.2 | 3017.23 | 75.15 |
| 19.5 | 2069.39 | 51.54 |
| 20.6 | 297.99 | 7.42 |
| 21.1 | 1679.9 | 41.84 |
| 21.8 | 638.46 | 15.90 |
| 22.4 | 157.34 | 3.92 |
| 23.2 | 1663.38 | 41.43 |
| 23.5 | 1054.12 | 26.25 |
| 25.1 | 1334.15 | 33.23 |
| 26.1 | 571.15 | 14.22 |
| 26.5 | 676.46 | 16.85 |
| 27.2 | 397.92 | 9.91 |
| 28.2 | 237.36 | 5.91 |
| 28.6 | 348.45 | 8.68 |
| 29.2 | 280.36 | 6.98 |
| 29.8 | 274.14 | 6.83 |
| 30.4 | 1004.43 | 25.02 |
| 31.1 | 76.65 | 1.91 |
| 31.8 | 187.02 | 4.66 |
| 32.5 | 60.39 | 1.50 |
| 33.4 | 89.71 | 2.23 |
| 34.3 | 110.17 | 2.74 |
| 37.4 | 100.36 | 2.50 |
| 38.9 | 23.93 | 0.60 |

### Example 3: Preparation of Compound (I) Crystal Form

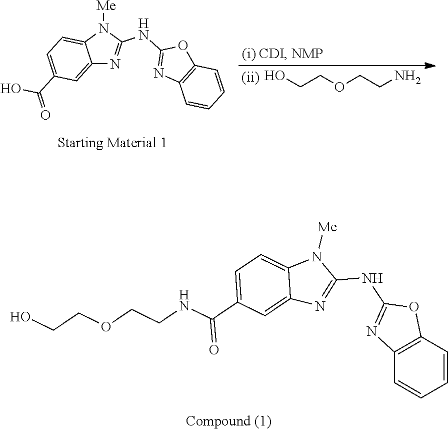 [SMILES: Cn1c(Nc2nc3ccccc3o2)nc2cc(C(=O)O)ccc21 ; NCCOCCO ; Cn1c(Nc2nc3ccccc3o2)nc2cc(C(=O)NCCOCCO)ccc21]

To a stirred slurry of 2-(benzo[d]oxazol-2-ylamino)-1-methyl-1H-benzo[d]imidazole-5-carboxylic acid (Starting Material 1; according to WO 2020/210339; 1215 g, 3.94 mol) in N-Methyl-2-pyrrolidone (NMP, 7.5 L) at 22° C. was added carbonyl diimidazole (CDI, 964 g). Additional NMP (3.7 L) was added. Reaction mixture was stirred at 45-49° C. (internal temperature) for 1.5 h. Internal temperature was lowered to 20-23° C., and 2-(2-aminoethoxy)ethan-lol (1224 g) was added dropwise using an addition funnel over a period of 15 minutes (reaction temperature increased to 31.5° C.). Addition funnel was rinsed with NMP (757 mL), and the wash was also added to the reaction mixture. Stirring was continued for additional 15 minutes (reaction temperature dropped to 22° C.). Water (15 L) was added using a diaphragm pump, and resulting light green slurry was heated to 59° C. (internal temperature) with stirring for 2.5 h. Stirring and heating was stopped and the reaction mixture left standing overnight at room temperature (23° C.). Reaction mixture was neutralized by adding 3N HCl (3.0 L) at a rate of 90 mL/minute. Water (30 L) was added. Let resulting reaction mixture (slurry) stand for 30 minutes. Reaction mixture was vacuum filtered, isolated solid was washed with water (4×3 L) and acetonitrile (1×4 L). Isolated solid was dried in a vacuum oven to a constant weight, to yield Compound (I) (1340 g). The Compound (I) Crystal Form thus obtained was characterized by XRPD and DSC. Its XRPD (Condition A) is shown in FIG. 9 and its DSC (Condition A) is shown in FIG. 10. XRPD peak listing is provided below in Table 5.

TABLE 5

| Pos. [°2θ] | Height [cts] | Rel. Int. [%] |
|---|---|---|
| 4.6 | 8292.89 | 100.00 |
| 9.2 | 626.76 | 7.56 |
| 13.6 | 273.46 | 3.30 |
| 13.9 | 537.85 | 6.49 |
| 14.3 | 1080.41 | 13.03 |
| 14.8 | 645.41 | 7.78 |
| 15.2 | 630.79 | 7.61 |
| 16.6 | 2016.19 | 24.31 |
| 17.4 | 1173.67 | 14.15 |
| 18.6 | 1686.4 | 20.34 |
| 19.4 | 213.63 | 2.58 |
| 19.9 | 695.47 | 8.39 |
| 21.4 | 1311.08 | 15.81 |
| 22.2 | 716.32 | 8.64 |
| 23.3 | 630.27 | 7.60 |
| 23.7 | 521.78 | 6.29 |
| 24.1 | 895.65 | 10.80 |
| 24.4 | 520.44 | 6.28 |
| 25 | 450.19 | 5.43 |
| 25.4 | 1715.06 | 20.68 |
| 26.7 | 512.59 | 6.18 |
| 27.2 | 696.79 | 8.40 |
| 27.9 | 1253.27 | 15.11 |
| 29.2 | 1097.74 | 13.24 |
| 30.8 | 102.86 | 1.24 |
| 32 | 106.99 | 1.29 |
| 32.8 | 424.56 | 5.12 |
| 33.6 | 141.23 | 1.70 |
| 35.1 | 77.66 | 0.94 |
| 35.9 | 120.03 | 1.45 |
| 36.6 | 156 | 1.88 |
| 37.5 | 33.4 | 0.40 |
| 38.8 | 66.55 | 0.80 |

### Example 4: Hygroscopicity

Dynamic vapor sorption (DVS) was performed with an ADVENTURE series DVS at 25° C. under nitrogen blow. Approximately 30 milligrams of material was used. Samples were analyzed using the methods below:
1. 0% RH to 95% RH at 10% RH (5% from 90 to 95% RH)
2. 95% RH to 0% RH at 10% RH (5% from 95 to 90% RH)
   The phosphate salt showed a weight gain up at 0.835% at 90% RH, whereas the citrate salt showed a weight gain up to 3.223% at 90% RH.

### Example 5: Thermodynamic Solubility

Thermodynamic solubility measurement was conducted in water and three bio-media (SGF, FaSSIF and FeSSIF) at 37° C. to understand the dissolution and disproportionation risk of two salt leads, using starting freebase as control. Specifically, about 40 mg samples (calculated with freebase) were added to 4.0 mL of each buffer. After shaking at 100 rpm at 37° C. for 1/4/24 hour(s), about 1.0 mL of each suspension was centrifuged for 5 minutes. The solids were analyzed by XRPD for crystal form, and the filtrates of supernatants were detected by HPLC for solubility and by pH meter for pH value. The results are shown below in Table 6.

TABLE 6

| | 1 Hr | | | 4 Hrs | | | 24 Hrs | | |
|---|---|---|---|---|---|---|---|---|---|
| | Solubility | | | Solubility | | | Solubility | | |
| Sample | (mg/mL) | pH | FC | (mg/mL) | pH | FC | (mg/mL) | pH | FC |

Thermodynamic Solubility in Water at 37° C.

| | | | | | | | | | |
|---|---|---|---|---|---|---|---|---|---|
| Citrate | 3.07 | 2.06 | Yes | 3.22 | 2.12 | NA | 1.64 | 2.33 | NA |
| Phosphate | 4.39 | 2.05 | Yes | 3.23 | 2.04 | NA | 2.06 | 1.97 | NA |
| Free form | ND | 5.69 | No | ND | 5.07 | No | ND | 5.95 | No |

Thermodynamic Solubility in SGF at 37° C.

| | | | | | | | | | |
|---|---|---|---|---|---|---|---|---|---|
| Citrate | >50.44 | 1.52 | Clear | >56.19 | 1.67 | Clear | >51.64 | 1.84 | Clear |
| Phosphate | >25.87 | 1.36 | Clear | >28.41 | 1.37 | Clear | >28.42 | 1.41 | Clear |
| Free form | 7.04 | 2.34 | No | 6.86 | 2.16 | No | 7.08 | 2.51 | No |

Thermodynamic Solubility in FaSSIF at 37° C.

| | | | | | | | | | |
|---|---|---|---|---|---|---|---|---|---|
| Citrate | 0.54 | 2.62 | Yes | 0.40 | 2.62 | NA | 0.05 | 2.63 | NA |
| Phosphate | 0.19 | 2.37 | Yes | 0.18 | 2.37 | NA | 0.07 | 2.36 | NA |
| Free form | ND | 6.30 | No | ND | 6.35 | No | ND | 5.99 | No |

Thermodynamic Solubility in FeSSIF at 37° C.

| | | | | | | | | | |
|---|---|---|---|---|---|---|---|---|---|
| Citrate | 0.26 | 3.51 | Yes | 0.22 | 3.59 | NA | 0.08 | 3.90 | NA |
| Phosphate | 1.40 | 4.06 | Yes | 0.77 | 4.21 | NA | 0.39 | 4.31 | NA |
| Free form | ND | 4.78 | No | ND | 4.73 | No | ND | 4.81 | No |

FC: Form Change
ND: Not Detected

It is apparent from Table 6 that the citrate and phosphate salts compared to the free form show significant solubility improvement in water and bio-relevant media.

### Example 6: Pharmacokinetic (PK) Studies

A PK Study of Crystalline form of 1:1 compound (I) Phosphate and compound (I) crystal form was conducted in Cynomolgus macaques (Cynomolgus non-human primates; Macaca fascicularis). The phosphate salt and compound (I) free form, were administered (10 and 10 mpk, PO, single dose) to Cynomolgus macaques (4 groups of 3 males each). Blood samples were collected at 0.25, 0.5, 1, 2, 4, 6, 8 and 24 hours, and were analyzed to determine Cₘₐₓ and exposure (AUC). The results are shown in Table 7.

TABLE 7
PK profile

| | Cₘₐₓ | AUCₗₐₛₜ | AUC_{inf} |
|---|---|---|---|
| Dose (mg/Kg) | (ng/ml) | (hr*ng/ml) | (hr*ng/ml) |
| 10 (free form) | 164 | 1925 | 2030 |
| 100 (free form) | 350 | 4079 | 4745 |
| 10 (phosphate salt) | 322 | 3476 | 3593 |
| 100 (phosphate salt) | 723 | 7578 | 9326 |

PK results in Table 7 show, the phosphate salt produces both Cₘₐₓ and exposure (AUC) roughly double compared to the free form.

1. A phosphate salt of Compound (I) represented by the following structural formula:

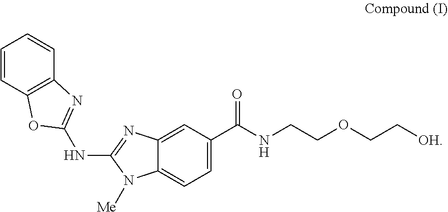 [SMILES: Cn1c(Nc2nc3ccccc3o2)nc2cc(C(=O)NCCOCCO)ccc21]

2. The phosphate salt of claim 1, wherein at least 90% by weight of the salt is crystalline.

3. The phosphate salt of claim 2, wherein at least 90% by weight of the salt is in a single crystal form.

4. The phosphate salt of claim 1, wherein the phosphate salt is a single crystal form characterized by the X-ray powder diffraction pattern shown in FIG. 1.

5. The phosphate salt of claim 1, wherein the phosphate salt is a single crystal form characterized by an X-ray powder diffraction pattern which comprises peaks (2θangles±0.2°) at 7.7°, 10.4° and 16.1° in 2θ.

6. The phosphate salt of claim 1, wherein the phosphate salt is a single crystal form characterized by an X-ray powder diffraction pattern which comprises peaks (2θangles±0.2°) at 7.7°, 8.9°, 10.4°, 13.0° and 16.1° in 20.

7. The phosphate salt of claim 1, wherein the phosphate salt is a single crystal form characterized by an X-ray powder diffraction pattern which comprises peaks (2θangles±0.2°) at 7.7°, 8.9°, 10.4°, 11.5°, 13.0°, 15.4°, 16.10, 17.5°, 17.9° and 21.7° in 20.

8. The phosphate salt of claim 1, wherein the phosphate salt is a single crystal form characterized by an X-ray powder diffraction pattern which comprises peaks (2θangles±0.2°) at 7.7°, 8.9°, 10.4°, 11.5°, 13.0°, 15.4°, 16.10, 17.5°, 17.9°, 19.1°, 19.4°, 20.4°, 21.7°, 23.0° and 24.9° in 2θ.

9. The phosphate salt of claim 5, wherein the X-ray powder diffraction pattern further comprises peaks (2θangles±0.2°) at 21.2°, 23.9°, 26.0° and/or 28.5° in 20.

10. The phosphate salt of claim 1, wherein the phosphate salt is a single crystal form characterized by a differential scanning calorimeter phase transition onset temperature of 239° C.±3° C.

11. The phosphate salt of claim 1, wherein the phosphate salt is a single crystal form characterized by the X-ray powder diffraction pattern shown in FIG. 3.

12. The phosphate salt of claim 1, wherein the phosphate salt is a single crystal form characterized by differential scanning calorimeter phase transition onset temperature between 227° C.±3° C. and 239° C.±3° C..

13. A pharmaceutical composition comprising the salt of claim 1, and a pharmaceutically acceptable carrier or diluent.

14. A method of increasing the activity of or the amount of HMOX-1 in a human subject comprising administering to the subject an effective amount of the salt of claim 1.

15. A method of activating transcription factor Nrf2 in a human subject comprising administering to the subject an effective amount of the salt of claim 1.

16. A method of reducing the amount of ROS in a human subject comprising administering to the subject an effective amount of the salt of claim 1.

17. A method of treating a disease, disorder, or condition in a human subject comprising administering to the subject an effective amount of the salt of claim 1, wherein the disease, disorder, or condition is: (i) a fibrotic disease, including a fibrotic disease of the lung, chronic obstructive pulmonary disease (COPD), idiopathic pulmonary fibrosis, sarcoidosis, a fibrotic disease of the liver including those caused by alcoholic cirrhosis, steatosis, cholestasis, drug side effect, and viral infection, a fibrotic diseases of the skin, scleroderma, or psoriasis; (ii) a neurodegenerative disease, including Friedreich's ataxia, Alzheimer's disease, Parkinson's disease, amyotrophic lateral sclerosis, multiple sclerosis, cerebral nerve degenerative disease, or Charcot-Marie-Tooth syndrome; (iii) a cardiovascular disease, including hypertension, hypercholesterolaemia, atherosclerosis, arteriosclerosis, thrombosis, acute coronary thrombosis, deep vein thrombosis, peripheral vascular disease, congestive heart failure, acute coronary syndrome, failure of arterial fistula for dialysis, ischemia-reperfusion injury, primary pulmonary hypertension, primary pulmonary arterial hypertension, or secondary pulmonary arterial hypertension; (iv) a renal disease, including acute kidney injury, polycystic kidney disease, Alport syndrome, diabetic nephropathy, glomerular nephritis, lupus nephritis, sickle cell nephropathy, and acute tubular necrosis; (v) an inflammatory disease, including asthma, chronic obstructive pulmonary disease, idiopathic pulmonary fibrosis, inflammatory bowel syndrome, Crohn's disease, celiac disease, ulcerative colitis, chronic inflammatory bowel disease, scleroderma, dermatitis, systemic lupus erythematosus, esophagitis, vasculitis, pancreatitis, tendonitis, osteoarthritis, rheumatoid arthritis, ankylosing spondylitis, or chronic inflammation of the brain; (vi) a liver disease, including drug induced liver toxicity, nonalcoholic steatohepatitis, hepatitis B infection, or hepatitis C infection; (vii) an eye disease, including conjunctivitis, glaucoma, uveitis, an eye wound, eye trauma, corneal grafts, Fuchs' endothelial corneal dystrophy, macular degeneration, cataracts, light retinopathy, retinitis pigmentosa, diabetic retinopathy, and retinopathy of prematurity; (viii) a thyroid disease, including Graves' disease, follicular adenoma, or papillary and follicular carcinomas; (ix) a viral infection, including infections from human immunodeficiency virus, hepatitis B, hepatitis C, or herpesvirus; (x) osteoporosis; (xi) a pregnancy disorder; (xii) endometriosis; (xiii) diabetes, including type 1 diabetes mellitus, type 2 diabetes mellitus, gestational diabetes, pre-diabetes, hyperglycemia, metabolic syndrome, or a secondary condition resulting from a diabetic condition; (xiv) cancer; (xv) a skin disease, including dermatitis, scleroderma, or psoriasis; (xvi) a mitochondrial diseases such as mitochondrial myopathies, Leber's hereditary optic neuropathy (LHON), myoclonic epilepsy with ragged red fibers (MERFF), mitochondrial encephalomyopathy, lactic acidosis and stroke-like episodes (MELAS) or Leigh's Syndrome; (xvii) a hematological disorder such as Diamond Blackfan anemia, myelodysplasic syndrome, sickle cell disease and beta-thalessemia; or (xviii) a muscle diseases, such as Duchenne muscular dystrophy, limb girdle muscular dystrophy, Becker muscular dystrophy, myotonic dystrophy and rhabdomyolysis.

18.-20. (canceled)
